(19) United States
(12) Patent Application Publication
(10) Pub. No.: US 2025/0098953 A1
(43) Pub. Date: Mar. 27, 2025

(54) URETEROSCOPE DEVICE AND SYSTEMS INCLUDING A CATHETER CONTROL SYSTEM, AND RELATED METHODS

(71) Applicant: C.R. BARD, INC., Murray Hill, NJ (US)

(72) Inventors: Justin Wolfe, Lawrenceville, GA (US); Vinothkannan Harikrishnan, Chennai (IN); Tamilvel Manickam, Chennai (IN)

(21) Appl. No.: 18/832,400

(22) PCT Filed: Feb. 1, 2022
(86) PCT No.: PCT/US2022/014777

§ 371 Date: Jul. 23, 2024

(51) Int. Cl. A61B 1/307; A61B 1/00; A61B 1/005; A61B 1/045; A61B 1/05; A61B 1/06

(52) U.S. Cl. CPC A61B 1/307; A61B 1/00018; A61B 1/0004; A61B 1/00042; A61B 1/00043; A61B 1/00114; A61B 1/00119; A61B 1/0052; A61B 1/0055; A61B 1/0057; A61B 1/045; A61B 1/05; A61B 1/0655; A61B 1/0676

(57) ABSTRACT

A ureteroscope system including a handpiece, a catheter, and a control system is described. The handpiece having a proximal end region, a distal end region opposite to the proximal end region, and an interior region. The catheter extends from the distal end region of the handpiece, and includes an active bend portion distal to the handpiece. The control system includes a sensor positioned in the interior region of the handpiece and configured to detect movement of the handpiece. The control system includes a motor positioned in the interior region of the handpiece and operably coupled to the sensor effective to activate the motor when the sensor detects movement of the handpiece. The ureteroscope system includes one or more wires secured to the active bend portion of the catheter and adjustable responsive to activation of the motor effective to at least partially bend the active bend portion of the catheter.

### BACKGROUND

Endoscopes of small size are desired in many industrial and medical applications. For example, when natural orifices and lumens of a human body are small, small endoscopes are used for insertion through such orifices and lumens to target locations within the body. For single incision laparoscopy, smaller endoscopes are preferred to provide an inside-the-body view of the surgical site, particularly when the incision itself is of minimal dimensions. Sometimes, patients may feel irritation when an endoscope is being inserted into his or her body, and a smaller endoscope may mitigate such unpleasant experience and may minimize trauma to the patient. Moreover, a physician may improve diagnostic and procedural protocols with a smaller endoscope. For example, transnasal endoscopy may sometimes replace trans-oral endoscopy. There is a need to provide a ureteroscope system that potentially allows for further minimizing trauma to a patient.

### SUMMARY

Embodiments disclosed herein are related to ureteroscope systems and methods of operating ureteroscope systems. In an embodiment, a ureteroscope system includes a handpiece, a catheter, and a control system. The handpiece has a proximal end region, a distal end region opposite to the proximal end region, and an interior region. The catheter extends from the distal end region of the handpiece, and includes an active bend portion distal to the handpiece. The control system includes a sensor positioned in the interior region of the handpiece and configured to detect movement of the handpiece, a motor positioned in the interior region of the handpiece and operably coupled to the sensor, the motor configured to be activated responsive to the sensor detecting movement of the handpiece, and one or more wires secured to the active bend portion of the catheter and adjustable responsive to activation of the motor effective to at least partially bend the active bend portion of the catheter.

In an embodiment, a method of operating a ureteroscope system is disclosed. The method includes inserting at least a distal end of a catheter of a ureteroscope into a patient, the distal end of the catheter including an image sensor and an active bend portion. The method also includes sensing, with a sensor positioned in an interior region of the handpiece, movement of the handpiece. The method also includes at least partially bending the active bend portion of the catheter in response to sensing the movement of the handpiece. The ureteroscope includes a motor positioned in the interior region of the handpiece and operably coupled to the sensor, the motor configured to be activated responsive to the sensor detecting movement of the handpiece, and one or more wires secured to the active bend portion of the catheter and adjustable responsive to activation of the motor effective to at least partially bend the active bend portion of the catheter

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A illustrates a ureteroscope as part of an endoscope system, according to an embodiment.

FIG. 1B illustrates an end view of a distal end of the catheter of the ureteroscope of FIG. 1A, according to an embodiment.

FIG. 2 is a side view of the handpiece of the ureteroscope of FIG. 1A with a side of the handpiece removed, according to an embodiment.

FIGS. 3A and 3B are side views of the ureteroscope of FIG. 1A showing automatic movement of an active bend portion of a catheter of the ureteroscope responsive and relative to movement of the handpiece of the ureteroscope, according to an embodiment.

FIG. 4 is an isometric view of a ureteroscope, according to an embodiment.

FIG. 5 is a schematic of a controller that may be used in systems and methods described herein, according to an embodiment.

FIG. 6 is a flow diagram of a method of operating a ureteroscope system, according to an embodiment.

### DETAILED DESCRIPTION

Embodiments disclosed herein relate to devices, systems, and methods of using an endoscope, such as a ureteroscope. The endoscope systems include a handpiece and a catheter. At least one, some, or all embodiments of systems and methods described herein include a control system built into the handpiece of the ureteroscope. The configuration of the control system may result in the technical effect of allowing a user to control an active bend portion of the catheter through movement of the handpiece. For example, the configuration of at least one, some, or all embodiments of the control systems described herein result in the technical effect of sensing acceleration (e.g., movement) of the handpiece and a processor of the system may use data or signals from the control system to provide the technical effect of determining data corresponding at least one of the direction and/or magnitude of movement or motion of the handpiece. At least one, some, or all embodiments of processors described herein then use this data corresponding to at least one of the direction and/or magnitude of movement of the handpiece to provide the technical effect of coordinating movement of one or more actuators (e.g., servos or linear actuators) to retract or release control wires through a mechanical mechanism. This retraction and/or release of the control wires results in the technical effect of bending or other movement of the active bend portion of the catheter. The combination of features of embodiments disclosed herein may lead to more smoothly advancing the catheter, and therewith to less trauma to the patient. In some embodiments described herein, the system may include a controller positioned on the handpiece that is configured to selectively activate and/or deactivate the control system.

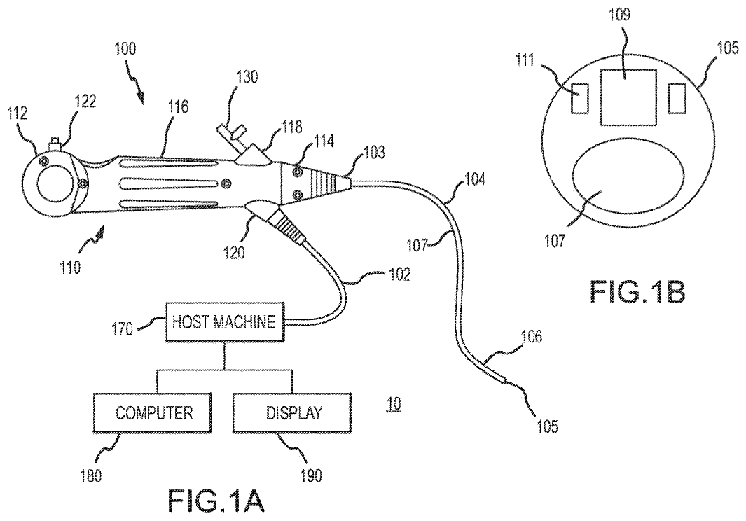

FIG. 1A illustrates an endoscopic system 10. In an embodiment, the endoscopic system 10 includes a digital ureteroscope 100 operably coupled to one or more electronic devices, such as a host machine 170 and one or more external terminals including a display 190 and a computer 180. The ureteroscope 100 may be operably coupled to one or more of the host machine 170, the computer 180, or the display 190 wirelessly or through a cable 102. The ureteroscope 100 includes a catheter 104 and a handpiece 110 having a catheter end (e.g., distal end region) 114 and a control end (e.g., proximal end region) 112 opposite to the catheter end 114. As the control end 112 is opposite to the catheter end 114, the control end 112 and the catheter end 114 are at different ends of the handpiece 110 and may face different directions relative to one another. In an embodiment, both the catheter 104 and the handpiece 110 are disposable. In some embodiments, the catheter 104 and the handpiece 110 are manufactured as an integral part, or the catheter 104 is fixed with the handpiece 110 via a handpiece-catheter connector 103. Alternatively, only the catheter 104 is disposable, and the handpiece 110 may be sterilized and reused multiple times. In this case, the catheter 104 is removably connected to the handpiece 110 and the catheter end 114 via a handpiece-catheter connector 103.

The catheter 104 of the ureteroscope 100 may be used for imaging an interior surface of a tubular structure, such as a lumen in the body of human or animal. For example, the catheter 104 may be inserted via a subject's urethra to access various parts of the urinary tract. However, it should be appreciated that the ureteroscope 100 may be employed as an industrial endoscope when tubular structure is a part of an industrial apparatus, an equipment, a product, a machine, a production line, and the like. In some embodiments, the catheter 104 may serve as a tether, and may include a plurality of scale markings or fiducials that enable a physician to measure a distance traveled by optoelectronic module into the tubular structure, such as a lumen of a body. Other structure(s) may be built into the ureteroscope 100 as desired.

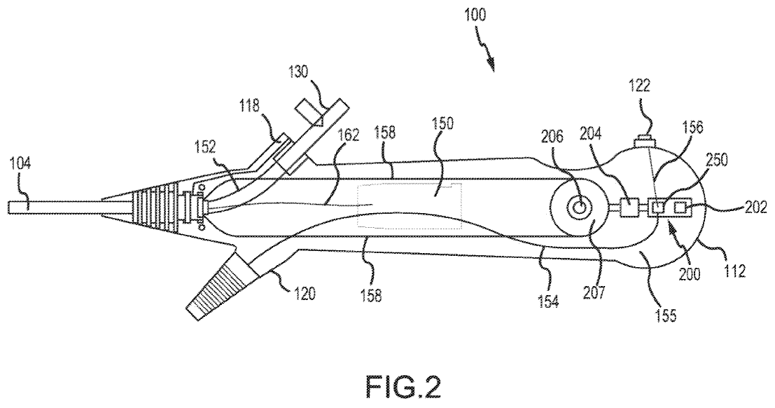

The catheter 104 also includes an active bend portion 106 at a distal end region 105 of the catheter 104. The active bend portion 106 is configured to selectively bend one or more directions. For example, the handpiece 110 may include a control system 200 (FIG. 2) configured to control one or more steering wires 158 (FIG. 2) that are connected to the active bend portion 106 of the catheter 104 to deflect the distal end 105 to the desired location. Accordingly, a user may selectively bend or curve the catheter 104 using the control system 200, described in greater detail below. In some embodiments, the active bend portion 106 of the catheter 104 is made of certain flexible medical-grade polymer materials to allow for steerability of the catheter and reduce discomfort to patients. The catheter 104 may include the active bend portion 106 and a passive bend portion 107. The active bend portion 106 may be generally distal to the handpiece 110 and may be controlled by one or more (e.g., two) control wires 158 (e.g., steel wires) to bend in two directions, one being used for bending the active bend portion 106 to one direction with a 0° to about 275° range, the other being used for bending the active bend portion 106 to the opposite direction with the same degree range. Other number of steering wires (e.g., 1, 3, or 4) may be implemented here as well. In contrast, the passive bend portion 107 may be between the active bend portion 106 and the handpiece 110 and may bend according to the shape of urinary tract during insertion. Suitable active bend portions of catheters are disclosed in U.S. Pat. No. 11,129,519 and U.S. Patent Publication No. 2018/0140177, the disclosures of which are incorporated herein, in their entirety, by this reference.

The ureteroscope 100 may include an optoelectronic module (e.g., a camera or other imager) for imaging the interior of the subject. For example, turning to FIG. 1B, an optoelectronic module 109 and at least one light source 111 may be located in a distal end 105 of the catheter 104 or other location in the catheter 104. The optoelectronic module 109 may include a micro camera module having an image sensor microchip, a set of micro lenses, and a micro illumination module. Suitable optoelectronic modules are disclosed in U.S. Pat. No. 9,942,452, which is incorporated herein, in its entirety, by this reference. In some embodiments, the optoelectronic module 109 may be positioned in a rigid or semi-rigid shell-like housing at the distal end 105 configured for insertion into the tubular structure for imaging its interior surface. For example, the optoelectronic module 109 may be inserted into a patient's body through a natural body orifice, such as the mouth, nose, urethra, bladder, vagina, or anus. The ureteroscope 100 may therefore have different configurations for use as a gastrointestinal, a colonoscope, endoscopic ultrasound (EUS), endoscopic retrograde cholangiopancreatography (ERCP), or other suitable application. Applications of the ureteroscope 100 include diagnostic observation associated with endometrial polyps, infertility, abnormal bleeding, and pelvic pain, and surgical procedure such as embryo growth arrest and uterine malformation etc.

The ureteroscope 100 may include a receiving device or communication interface 150 (FIG. 2) generally located outside the catheter 104 for receiving the signal from an image sensor within the optoelectronic module 109. For example, the catheter 104 may include at least one electrical lead 162 (FIG. 2) that is coupled to the optoelectronic module 109 and conveys an electrical signal from optoelectronic module 109 to the communication interface 150. The communication interface 150 may be positioned within or adjacent to the handpiece 110.

The catheter 104 may be configured to couple the optoelectronic module 109 to the circuitry within the handpiece 110 in any suitable manner. For example, the availability of low-cost modular imaging system components enables the manufacture of a disposable components of the ureteroscope 100 at very low cost. In an embodiment, the catheter 104 is configured to detachably couple the optoelectronic module 109 to circuitry within the handpiece 110. In this manner, the catheter 104 and the optoelectronic module 109 are disposable, and may be detached from the handpiece 110 after a single patient use, thus eliminating the need for sterilization or reprocessing and reducing contamination risks. The handpiece 110 may be disinfected for subsequent reuse with a catheter 104 and optoelectronic module 109 for a different patient.

Returning to FIG. 1A, the endoscopic system 10 may include one or more electronic devices for processing and displaying the image data received from the optoelectronic module 109 of the ureteroscope 100. For example, the endoscopic system 10 may include one or more of a host machine 170 having a microprocessor, a computer 180 having a microprocessor, and a display 190. The host machine 170 may be connected to one or more terminals of the computer 180 and the display 190 for further processing and displaying the image data from the optoelectronic module 109. The host machine 170 or the computer 180 may be programmed with image processing software that takes as input the image data output from the optoelectronic module 109 of the ureteroscope 100 and generates two-or three-dimensional reconstructions of the body lumen that may be displayed on the display 190. Accordingly, a processor in at least one of the host machine 170, the computer 180, or the display 190 may be programmed with software that accepts as input a plurality of still images of an object generated by the optoelectronic module 109, and then output for display a three-dimensional rendering of the object based on the plurality of still images. The display 190 may include any suitable display, and may be configured to display a moving image (movie) or a still image collected by the image sensor of the optoelectronic module 109. Although shown in FIG. 1A as separate blocks, the host machine 170, the computer 180, and the display 190 may include a single device, two devices, three devices, or more than three devices.

The endoscopic system 10 also may include a cable 102 configured to operably couple the ureteroscope to at least one of the host machine 170, the computer 180, or the display 190. The cable 102 may electrically couple the communication interface 150 (FIG. 2) of the ureteroscope 100 to at least one of the host machine 170, the computer 180, or the display 190. The cable 102 also may allow the communication interface 150 to communicate with and receive electric power from the host machine 170 or other power sources. The cable 102 also may be configured to allow the communication interface 150 to transmit image data captured at the optoelectronic module 109 to the host machine 170 for processing, storing, and displaying.

Turning to FIG. 2, which illustrates a side view of the handpiece 110 with a portion of the handpiece 110, the ureteroscope 100 may include a communication interface 150 or host interface housed in the handpiece 110. The communication interface 150 may contain, for example, one or more of a processor board, a camera board and frame grabber, or a power source. The processor board may be coupled by the cable 102 to the host machine 170 for storage and retrieval of images generated by ureteroscope 100. The communication interface 150 also may be configured to communicate with and receive electric power from the host machine 170 or other power source via the cable 102. The communication interface 150 also may transmit image data captured at the distal end 105 to the host machine 170 for processing, storing, and displaying. The communication interface 150 may be connected to the cable 102 through one or more wires 154 and/or connected to the optoelectronic module 109 through one or more electrical leads 162.

Alternatively or in addition, the communication interface 150 or handpiece 110 may include an antenna and a wireless chipset, e.g., compliant with the IEEE 802.11 WiFi standards, for wirelessly transmitting the video or still image generated by the ureteroscope 100 to the host machine 170, the computer 180, or the display 190 without the cable 102. For example, the communication interface 150 may include a wireless interface configured to implement various protocols, including but not limited to Wi-Fi, Bluetooth, ZigBee, Z-Wave, etc. This arrangement may be useful in a physician's office because it permits the computer and display to be placed outside of the sterile field, while also allowing the physician greater maneuverability during use of the ureteroscope 100. In other embodiments, the communication interface 150 may be omitted and the image data may be transmitted directly to the host machine 170 and/or the computer 180.

The ureteroscope 100 may include one or more controls 122 positioned at or proximate to the control end 112 of the handpiece 110. In some embodiments, at least one of the one or more controls 122 are positioned between the control end 112 and the catheter end 114. For example, at least one (e.g., all) of the one or more controls 122 may be positioned closer to the catheter end 114 than the control end 122, such as proximate to the working channel port 118. The one or more controls 122 may include one or more of a switch, a button, a rotatable knob, a movable tab, and the like. The one or more controls may be electrically coupled to the communication interface 150 through one or more wires 154, 156. In some embodiments, the one or more controls 122 are configured to adjust views presented on the display 190. For example, the one or more controls may be configured to adjust at least one of a brightness, a zoom, a focus or a contrast of one or more images displayed on the display 190. Accordingly, the one or more controls 122 allow a user to adjust views presented on the display 190 and/or computer 180 according to the user's preference and as necessary during use of the ureteroscope 100.

The ureteroscope 100 may include one or more wires coupling the one or more controls 122 to the optoelectronic module 109 and/or the control system 200 to allow a user to adjust at least one of the brightness, the zoom, the focus, or the contrast on the optoelectronic module 109. For example, at least one of the one or more controls 122 may be connected to the optoelectronic module 109 through one or more wires connected to the communication interface 150 and/or the control system 200 and at least one of the one or more control 122 and an electric lead 162 connected to the communication interface 150 and the optoelectronic module 109. In some embodiments, one or more wires or electric leads may be connected directly to the optoelectronic module 109 and at least one of the one or more control 122. Coupling the one or more controls 122 to the communication interface 150 may allow a user to adjust at least one of the brightness, the zoom, the focus, or the contrast on the display 190, either wirelessly or through the cable 102.

In some embodiments, a processor of the host machine 170 or another processor coupled to the ureteroscope 100 is configured to display one or more view settings on the display 190. The one or more view settings may display responsive actuation of the one or more controls 122 or, alternatively, an additional button on the handpiece 110 or display 190. The one or more view settings displayed on the display 190 and/or computer 180 may include at least one of brightness, zoom, contrast, or focus. For example, responsive to actuation of at least one of the one or more controls 122, a brightness setting may be displayed on the display 190. After the brightness setting is displayed on the display 190, a user may adjust the brightness of the one or more images displayed on the display 190 using at least one of the one or more controls. Other view settings may be similarly displayed on the display 190 and adjusted by the user with the one or more controls 122.

In some embodiments, the one or more view settings also may be displayed on a processor display on the handpiece 110 or the host machine 170.

In some embodiments, at least one of the one or more controls 122 is configured to activate (e.g., turn on) or deactivate (e.g., turn off) at least one light source 111 (FIG. 1B) at the distal end 105 of the catheter 104. For example, the light source 111 may include a light-emitting diode (LED) positioned in the distal end 105 of the catheter. Alternatively or in addition, the ureteroscope 100 may include a LED light source positioned elsewhere, such as in the handpiece 110, which provides illumination to the at least one light source 111 at the distal end 105 via one or more sets of optical fibers extending through the catheter 104. In some instances of use, it is desirable to turn the at least one light source 111 of the ureteroscope 100 completely off during backloading of the ureteroscope 100 over guidewires, rather than merely incrementally adjusting the light. Accordingly, at least one of the one or more controls allows a user to selectively turn off and turn on the light source 111 when desired by the user.

In some embodiments, at least one of the one or more controls 122 is configured to activate and deactivate the optoelectronic module 109. At least one of the one or more controls 122 also may be configured to switch an image mode in at least one of the optoelectronic module 109, the retrieving device 150, or the one or more electronic devices between a still image mode whereby a still image is recorded and a video image mode whereby a video stream is recorded. At least one of the one or more controls 122 also may be configured to activate a frame grabber mode in at least one of the optoelectronic module 109, the retrieving device 150, or the one or more electronic devices that creates a still image from the video stream output generated by the optoelectronic module 109. For example, at least one of the one or more controls 122 may be configured to communicate with one or more of the host machine 170, the computer 180, or the display 190 to allow a user to create a still image from a video stream being displayed on the display 190, and storing or recording the still image on at least one of the host machine 170 or the computer 180.

In some embodiments, at least one of the one or more controls 122 is configured to activate and deactivate the control system 200. In at least one, some or all embodiments, at least one of the one or more controls 122 activate the control system 200 to provide the technical result of allowing a user to control the active bend portion 106 by moving the handpiece 110 and at least one of the one or more controls 122 may deactivate the control system 200 such that movement of the handpiece 110 does not control the active bend portion 106. In at least one, some, or all embodiments, at least one of the one or more controls activate the control system 200 to provide the technical effect of allowing a user to control at least one of brightness, zoom, contrast, or focus by moving the handpiece 110 and at least one of the one or more controls 122 may deactivate the control system 200 such that movement of the handpiece 110 does not control at least one of brightness, zoom, contrast, or focus. In at least one, some, or all embodiments, at least one of the one or more controls 122 activate the control system 200 to provide the technical effect of allowing a user to control switching between a still image mode and a video image mode by moving the handpiece 110 and at least one of the one or more controls 122 may deactivate the control system 200 such that movement of the handpiece 110 does not control switching between a still image mode and a video image mode. In at least one, some, or all embodiments, at least one of the one or more controls 122 activate the control system 200 to provide the technical effect of allowing a user to control activation and deactivation of the at least one light source 111 by moving the handpiece 110 and at least one of the one or more controls 122 may deactivate the control system 200 such that movement of the handpiece 110 does not control activation and deactivation of the at least one light source 111. In at least one, some, or all embodiments, at least one of the one or more controls 122 activate the control system 200 to provide the technical effect of allowing a user to activate a frame grab to record a still image during a video stream mode by moving the handpiece and at least one of the one or more controls 122 may deactivate the control system 200 such that movement of the handpiece does not activate a frame grab during a video stream mode.

The ureteroscope 100 may be operated to perform or complete selected tasks manually, automatically, or a combination thereof. Some ureteroscopic functions may be implemented with the use of components that comprise hardware, software, firmware or combinations thereof. While general-purpose components such as general purpose computers or oscilloscopes may be used in the ureteroscope 100, dedicated or custom components such as circuits, integrated circuits or software may be too. For example, some functions are implemented with a plurality of software instructions executed by one or more data processors, which is part of a general-purpose or custom computer. The one or more data processors may be in at least one of the communication interface 150, the host machine 170, the computer 180, or the display 190. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more input devices (e.g., allowing input of commands and/or parameters) and output devices (e.g., allowing reporting parameters of operation and results).

The handpiece 110 also includes a working channel port 118 and a working channel opening 107 (FIG. 1B) that provides fluid communication between the working channel port 118 and the distal end 105 of the catheter 104, according to an embodiment. The handpiece 110 also may include a working channel connector 130 that is connected or attached to the working channel port 118 and a working channel 152 (FIG. 2) that provides fluid communication between working channel port 118 and the working channel opening 107 through the handpiece 110 and/or the catheter 104. The working channel port 118, the working channel connector 130, and the working channel 152 are optional and may be absent from some embodiments. The working channel 152 may include a single, continuous material through the handpiece 110 and the catheter 104. In some embodiments, the working channel 152 includes a first portion in the interior region 155 of the handpiece 110 and a second portion in the catheter 104. The first portion and the second portion of the catheter 152 may include different materials or configurations that are fluidly connected to one another. At least one of the working channel port 118 or the working channel connector 130 is configured to engage with various surgical instruments and irrigation devices, as needed, for operations such as stone breaking and retrieval, etc. The working channel port 118 may be positioned on a lower/bottom or an upper/top portion of the handpiece 110, proximate to the catheter end 114 of the handpiece 110. The working channel port 118 may be positioned less than one-half of a distance from the catheter end 114 to the control end 112, less than one-third of the distance from the catheter end 114 to the control end 112, less than one-quarter of the distance from the catheter end 114 to the control end 112, or less than one-fifth of the distance from the catheter end 114 to control end 112.

The handpiece 110 also may include a cable port 120 for receiving or connecting the cable 102 (FIGS. 1A), thereby operably coupling the ureteroscope 100 to at least one of the host machine 170, the computer 180, or the display 190. Both the cable port 120 and the working channel port 118 also may be positioned on a lower or bottom portion of the handpiece 110, proximate to the catheter end 114 of the handpiece 110. For example, both the cable port 120 and the working channel port 118 may be positioned less than one-half of a distance from the catheter end 114 to the control end 112, less than one-third of the distance from the catheter end 114 to the control end 112, less than one-quarter of the distance from the catheter end 114 to the control end 112, or less than one-fifth of the distance from the catheter end 114 to control end 112. Alternatively, the handpiece 110 may further include a compact battery module for supplying power to the optoelectronic module 109 and the at least one light source 111. The power source in the handpiece 110 may be, for example, one or more conventional dry-cell disposable batteries or lithium ion rechargeable batteries.

Moreover, while the ureteroscope 100 shown in FIGS. 1-3 includes a cable port 120 positioned proximate to the catheter end 114 and distal to the control end 112, in some embodiments, the cable port 120 is absent from the handpiece 110 or positioned proximate to the control end 112 and distal to the catheter end 114. Accordingly, in some embodiments, the cable port 120 is absent from the handpiece 110 or positioned proximate to the control end 112, but the handpiece 110 includes one or more controls 122 positioned at the control end 112 of the handpiece 110 and a working channel port 118 disposed on the rounded first surface along a longitudinal central plane proximate to the catheter end 114. In still other embodiments, the one or more controls 122 are absent from the handpiece 110 and the cable port 120 is absent from the handpiece 110 or positioned proximate to the control end 112, but the handpiece includes a working channel port 118 disposed on the rounded first surface along the longitudinal central plane proximate to the catheter end 114.

Furthermore, while the ureteroscope 100 shown in FIGS. 1-3 includes a working channel port 118 disposed on the rounded first surface along the longitudinal central plane proximate to the catheter end 114 and distal to the control end 112, in some embodiments, the working channel port 118 may be disposed elsewhere on the handpiece 110. Accordingly, in some embodiments, the working channel port 118 may be disposed elsewhere than along the longitudinal central plane proximate to the catheter end 114, but the handpiece may include one or more controls 122 positioned at the control end 112 of the handpiece 110 and a cable port 120 positioned proximate to the catheter end 114. In still other embodiments, the one or more controls 122 are absent from the handpiece 110 and the working channel port 118 may be disposed elsewhere than along the longitudinal central plane proximate to the catheter end 114, but the handpiece 110 includes a cable port 120 positioned proximate to the catheter end 114. In some embodiments, the cable port 120 is absent from the handpiece 110 or positioned proximate to the control end 112 and the working channel port 118 may be disposed elsewhere than along the longitudinal central plane proximate to the catheter end 114, but the handpiece 110 includes the one or more controls 122 positioned at the control end 112 of the handpiece 110.

Turning now to FIG. 2, the endoscopic system 10 may include the control system 200. In some embodiments, the control system 200 is primarily a catheter control system configured to control bending of the active bend portion 106 of the catheter 104 responsive to movement or motion of the handpiece 110. The control system 200, may additionally or alternatively control other aspects of the endoscopic system 10 responsive to movement or motion of the handpiece 110, such as activation and/or deactivation of the light source 111, activation and/or deactivation of the image sensor of the optoelectronic module 109, changing of an image collection mode of the image sensor from a still image mode to a video stream mode, activation of a frame grab to record a still image during the video stream mode, adjusting zoom of the image sensor, deploying a tool in the working channel 152, and/or adjusting at least one of a brightness, a zoom, a focus, or a contrast of the one or more images from the image sensor displayed on a display 190.

In some embodiments, the control system 200 includes at least a sensor 202 positioned in the interior region 155 of the handpiece 110. The sensor 202 may be configured detect at least one (e.g. all) of movement of the handpiece 110, direction of movement of the handpiece 110, and/or magnitude of movement of the handpiece 110. In some embodiments, the sensor 202 includes at least one of an accelerometer, a MEMS sensor, and/or a gyroscope. In some embodiments, the sensor 202 includes any sensor configured to detect at least one (e.g. all) of movement of the handpiece 110, direction of movement of the handpiece 110, and/or magnitude of movement of the handpiece 110.

In some embodiments, the sensor 202 is configured to detect at least directional movement of at least a portion of the handpiece 110. For example, the sensor 202 may be configured to detect directional movement of a portion (e.g., the control end 112) of the handpiece 110 in at least two directions, such as a first handpiece direction and a second handpiece direction different from the first handpiece direction. More particularly, the sensor 202 may be configured to detect directional movement of a portion (e.g., the control end 112) of the handpiece 110 in upward and downward directions and/or or right and left directions relative to a starting point of the portion of the handpiece 110. In some embodiments, the sensor 202 is configured to detect direction movement of a portion (e.g., the control end 112) of the handpiece 110 in more than two directions. For example, the sensor 202 may be configured to detect movement of a portion (e.g., the control end 112) of the handpiece 110 on an x, y, z Cartesian coordinate system.

In some embodiments, the sensor 202 is configured to detect at least a magnitude of movement or motion of at least a portion of the handpiece 110. For example, the sensor 202 may be configured to detect how far and/or how quickly a portion (e.g., the control end 112) of the handpiece 110 moved in a direction relative to an initial starting point. More particularly, the sensor 202 may be configured to detect how far and/or how quickly (e.g., the rate at which) a portion (e.g., the control end 112) of the handpiece 110 moved in a first handpiece direction relative to an initial starting point and also how far and/or how quickly the portion of the handpiece 110 moved in a second handpiece direction relative to the initial starting point of the portion of the handpiece 110.

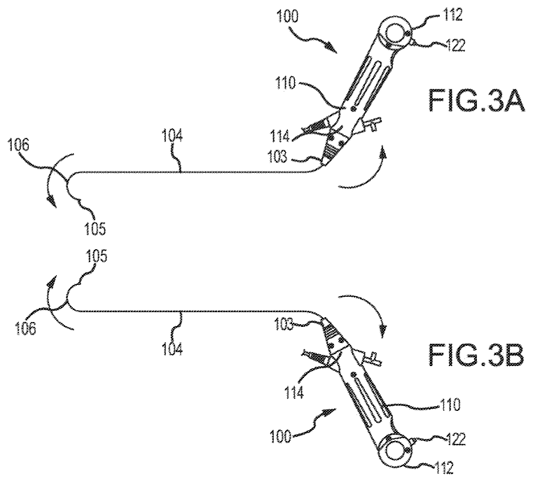

In at least one, some, or all embodiments, the sensor 202 is positioned in the interior region 155 at least proximate to the control end region 112 (e.g., proximal end region) of the handpiece 110. Positioning the sensor 202 proximate to or in the control end region 112 of the handpiece 110 results in the technical effect of allowing the sensor to detect movement or motion of the control end region 112 as a user to pivots the control end region 112 about an axis defined approximately by the catheter end 114 of the handpiece 110. The catheter end 114 of the handpiece 110 may remain substantially motionless or have less movement than the control end region 112, thus avoiding potential discomfort of the subject by movement of the catheter end 114 and the catheter 104 when moving the handpiece 110. FIGS. 3A-3B, for example, illustrate on the control end 112 may be pivoted about an axis defined approximately by the catheter end 114 and/or the handpiece- catheter connector 103 to move the active bend portion 106 on the catheter 104 while the catheter end 114 and/or the handpiece-catheter connector 103 remain substantially motionless or having less movement than the control end 112.

The control system 200 also includes a processor (e.g., microprocessor) 250 configured to communicate with at least the sensor 202. In the ureteroscope 100, the processor 250 is positioned in the control end 112 proximate to the sensor 202. In some embodiments, the processor 250 is positioned elsewhere in the interior region 155 of the handpiece 110, such as included with the communication interface 150. In some embodiments, the processor 250 may be included with one or more of the host machine 170, the computer 180, and/or the display 190.

The processor 250 is configured to receive data or signals from the sensor 202, according to an embodiment. For example, the processor 250 may be configured to receive data from the sensor 202 pertaining at least in part to at least one (e.g. all) of movement of the handpiece 110, direction of movement of the handpiece 110, and/or magnitude of movement of the handpiece 110 detected by the sensor 202, as described above in greater detail. The processor 250 may be configured to use the data received from the sensor 202 to determine at least one (e.g. all) of movement of the handpiece 110, direction of movement of the handpiece 110, and/or magnitude of movement of the handpiece 110. The processor 250 is configured to use this data received from the sensor 202 to coordinate movement of the active bend portion 106 responsive to movement of at least a portion of the handpiece 110, according to an embodiment. The processor 250 may be configured to use this data received from the sensor 202 to coordinate, responsive to movement of at least a portion of the handpiece 110, activation and/or deactivation of the light source 111, activation and/or deactivation of the image sensor of the optoelectronic module 109, changing of an image collection mode of the image sensor from a still image mode to a video stream mode, activation of a frame grab to record a still image during the video stream mode, adjusting zoom of the image sensor, deploying a tool in the working channel 152, and/or adjust at least one of a brightness, a zoom, a focus, or a contrast of the one or more images from the image sensor displayed on a display 190.

In some embodiments, the processor 250 is configured to use the data received from the sensor 202 to program or otherwise instruct one or more actuators to move of the active bend portion 106 responsive to movement of at least a portion of the handpiece 110. For example, the ureteroscope may include a servo actuator and/or a linear configured to move the active bend portion 106 responsive to movement of at least a portion of the handpiece 110. In some embodiments, the control system 200 includes a motor 206 positioned in the interior region of the handpiece 110 and operably coupled to the sensor 202 and/or the processor 250, according to an embodiment. The processor 250 may be configured to activate the motor 206 when the sensor 202 detects movement of the handpiece 110. For example, the motor 206 may be positioned in the interior region 155 of the handpiece 110 and operably coupled to the sensor 202, with the motor 206 being configured to be activated responsive to the sensor 202 detecting movement of the handpiece 110. In some embodiments, the control system 200 includes a driver 204 operably coupled to the processor 250 and the motor 206. In some embodiments, the processor 250 is configured to coordinate movement of the driver 204 effective to adjust or move the motor 206 and move the active bend portion 106 responsive to movement of at least a portion of the handpiece 110. In some embodiments, the driver 204 includes a stepper driver and the motor 206 includes a stepper motor.

The ureteroscope 100 also may include the one or more wires 158 secured to the active bend portion 106 of the catheter 104 and adjustable (e.g., movable or tightened) responsive to activation of the motor 206 effective to at least partially bend the active bend portion 106 of the catheter 104. The one or more wires 158 may be secured directly to the motor 206 or may be secured to one or more components (such as a wire wheel 207) directly or indirectly secured to the motor 206. The one or more wires 158 are secured to the active bend portion 106 such that when the motor 206 turns or rotates a first direction, a first wire (or a first portion) of the one or more wires 158 pulls the active bend portion 106 towards a first bending direction, and when the motor 206 turns or rotates a second (or opposite) direction, a second wire (or a second portion) of the one or more wires 158 pulls the active bend portion 106 towards a second (or opposite) direction. In some embodiments, the one or more wires 158 are configured to move or bend the active bend portion 106 in more than two directions, such as move or bend the active bend portion 106 on an x, y, z Cartesian coordinate system corresponding to movement of a portion (e.g., the control end 112) of the handpiece on an x, y, z Cartesian coordinate system.

The degree of the bend in the active bend portion 106 in either the first bending direction or the second bending direction may be determined by the extent of the rotation of the motor 206 in the first direction or the second direction, respectively. In some embodiments, when the motor 206 turns or rotates a first direction, a first wire (or a first portion) of the one or more wires 158 is tightened effective to pull the active bend portion 106 towards a first bending direction, and when the motor 206 turns or rotates a second (or opposite) direction, a second wire (or a second portion) of the one or more wires 158 is tightened effect to pull the active bend portion 106 towards a second (or opposite) bending direction. In some embodiments, the motor 206 includes a wire wheel 207 rotatable in a first direction and a second (or opposite) direction, and the one or more wires are secured directly to the wire wheel 207.

Turning now to FIGS. 3A-3B, which illustrate movement of the active bend portion 106 responsive to movement of at least a portion of the handpiece 110, according to an embodiment. To use and operate the control system 200, a user (e.g. ureteroscope operator) may selectively activate the control system 200 using at least one of the one or more controls 122 on the handpiece 110. For example, the user may press, rotate, turn, flip or otherwise adjust at least one of the one or more controls 122 to activate the control system 200. When the control 122 activates the control system 200, the motor 206 adjusts the one or more wires 158 responsive to the sensor 202 detecting movement of the handpiece 110.

For example, turning specifically to FIG. 3A, with the control system 200 activated, a user may move the control end 112 in a first handpiece direction (such as upwards in FIG. 3A). The sensor 202 in the control end 112 detects movement of the control end 112 in the first handpiece direction, and the processor 250 receives data from the sensor 202 pertaining to the movement of the control end 112 in the first handpiece direction. Based on this data from the sensor 202, the processor 250 coordinates movement of the driver 204 and the motor 206 effective resulting in the technical effect of moving, adjusting, or tightening at least a first portion or a first wire of the one or more wires 158. This movement, adjustment, or tightening of the first portion or the first wire of the one or more wires 158 at least partially bends the active bend portion 106 in a first bend direction. As illustrated in FIG. 3A, in some embodiments, the first handpiece direction of movement of the control end 112 is generally opposite to the first bending direction of bending of the active bend portion 106. In some embodiments, the first handpiece direction of movement of the control end 112 is generally the same as the first bending direction of bending of the active bend portion 106. As described above, the sensor 202 also may detect a magnitude (e.g., amount and/or rate) of movement of the control end 112, and the processor 250 receives data from the sensor 202 pertaining to the magnitude of movement of the control end 112 in the first handpiece direction. Based on this data from the sensor 202, the processor 250 coordinates a magnitude of movement of the driver 204 and the motor 206 effective resulting in the technical effect of moving, adjusting, or tightening at least a first portion or a first wire of the one or more wires 158 correlating to the magnitude of movement of the control end 112 in the first handpiece direction. For example, moving or pivoting the control end 112 a first amount in the first handpiece direction bends the active bend portion 106 a first amount, and moving or pivoting the control end 112 a second amount in the first handpiece direction that is greater than the first amount bends the active bend portion 106 a second amount that is greater than the first amount of bend in the active bend portion 106.

Turning specifically to FIG. 3B, with the control system 200 activated, a user may move the control end 112 in a second handpiece direction (such as downwards in FIG. 3B). The sensor 202 in the control end 112 detects movement of the control end 112 in the v direction, and the processor 250 receives data from the sensor 202 pertaining to the movement of the control end 112 in the second handpiece direction. Based on this data from the sensor 202, the processor 250 coordinates movement of the driver 204 and the motor 206 effective to result in the technical effect of moving, adjusting, or tightening at least a second portion or a second wire of the one or more wires 158. This movement, adjustment, or tightening of the second portion or the second wire of the one or more wires 158 at least partially bends the active bend portion 106 in a second bending direction. As illustrated in FIG. 3B, in some embodiments, the second handpiece direction of movement of the control end 112 is generally opposite to second bending direction of bending of the active bend portion 106. In some embodiments, the second handpiece direction of movement of the control end 112 is generally the same as the second bending direction of bending of the active bend portion 106. As described above, the sensor 202 also may detect a magnitude (e.g., amount and/or rate) of movement of the control end 112, and the processor 250 receives data from the sensor 202 pertaining to the magnitude of movement of the control end 112 in the second handpiece direction. Based on this data from the sensor 202, the processor 250 coordinates a magnitude of movement of the driver 204 and the motor 206 effective to result in the technical effect of moving, adjusting, or tightening at least a second portion or a second wire of the one or more wires 158 correlating to the magnitude of movement of the control end 112 in the second handpiece direction. For example, moving or pivoting the control end 112 a first amount in the second handpiece direction bends the active bend portion 106 a first amount, and moving or pivoting the control end 112 a second amount in the second handpiece direction that is greater than the first amount bends the active bend portion 106 a second amount that is greater than the first amount of bend in the active bend portion 106. When a user deactivates the control system 200 using at least one of the one or more controls 122, the motor 206 does not adjust the one or more wires 158 responsive to the sensor 202 detecting movement of the handpiece 110 or the sensor 202 does not detect movement of the handpiece 110.

In use, at least one of the one more controls 122 may be configured to activate the control system 200 for control of a feature different than the activate bend portion 106 of the catheter 104. For example, the configuration of at least one of the one or more controls 122 may result in the technical effect of allowing at least one of the one or more controls 122 to activate the control system 200 for control of one or more of the light source 111, the image sensor of the optoelectronic module 109, the image collection mode of the image sensor, a frame grab, zoom of the image sensor, tool deployment in the working channel 152, and/or brightness, a zoom, a focus, or a contrast of the one or more images from the image sensor displayed on a display 190. In some embodiments, the one or more controls 122 include multiple controls, with a different control of the multiple controls configured to activate the control system 200 to control a different one of the active bend portion 106 of the catheter, the light source 111, the image sensor of the optoelectronic module 109, the image collection mode of the image sensor, a frame grab, zoom of the image sensor, tool deployment in the working channel 152, and/or brightness, a zoom, a focus, or a contrast of the one or more images from the image sensor displayed on a display 190.

In some embodiments, with the control system 200 activated for the one or more light sources 111, a user may move the control end 112 in a first handpiece direction to provide the technical effect of activating the one or more light sources 111 (without bending the active bend portion 106) and the user may move the control end 112 in a second handpiece direction to provide the technical effect of deactivating the one or more light sources 111. With the control system 200 activated for the image sensor of the optoelectronic module 109, a user may move the control end 112 in a first handpiece direction to provide the technical effect of activating the image sensor of the optoelectronic module (without bending the active bend portion 106) and the user may move the control end 112 in a second handpiece direction to provide the technical effect of deactivating the image sensor of the optoelectronic module, according to an embodiment. With the control system 200 activated for the image collection mode of the endoscopic system 10, a user may move the control end 112 in a first handpiece direction to provide the technical effect of changing the image collection mode of the image sensor to a still image mode (without bending the active bend portion 106) and may move the control end 112 in a second handpiece direction to provide the technical effect of changing image collection mode to a video stream mode, according to an embodiment. With the control system 200 activated for the frame grab, a user may move the control end 112 in either the first handpiece direction or the second handpiece direction to provide the technical effect of recording a still image during the video stream mode (without bending the active bend portion 106), according to an embodiment. With the control system 200 activated for tool deployment, a user may move the control end 112 in a first handpiece direction to provide the technical effect of deploying a tool in the working channel 152 (without bending the active bend portion 106) and may move the control end 112 in a second handpiece direction to retract the tool into the working channel 152, according to an embodiment. With the control system 200 activated for display control, a user may move the control end 112 in a first handpiece direction to provide the technical effect of increasing at least one of brightness, zoom, contrast and/or focus of the one or more images from the image sensor displayed on the display 190 (without bending the active bend portion 106) and may move the control end 112 in a second handpiece direction to provide the technical effect of decreasing at least one of brightness, zoom, contrast, and/or focus of the one or more images from the image sensor displayed on the display 190, according to an embodiment.

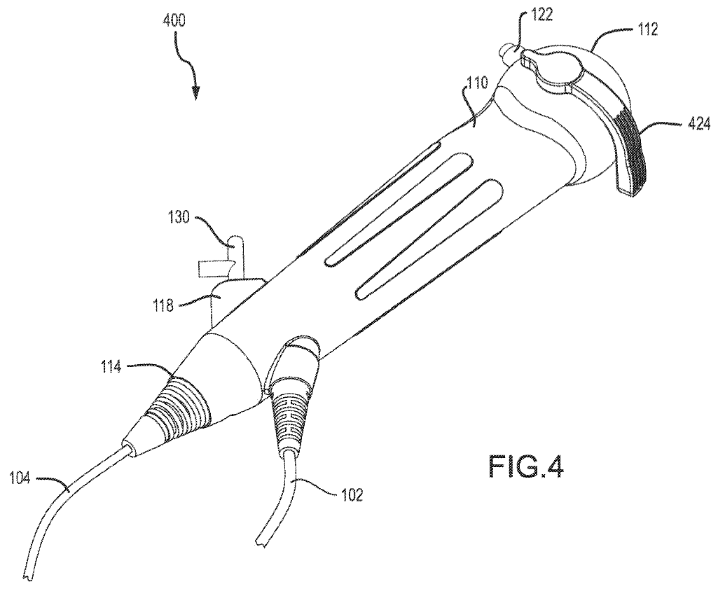

Turning now to FIG. 4, in some embodiments, a ureteroscope 400 may include a steering controller 424 secured to the handpiece 110 and positioned at least partially outside the interior region 155 of the handpiece 110. The steering controller 424 may be operably secured to the wire wheel 207 and selectively movable effective to move the wire wheel 207 and adjust the one or more wires 158 secured to the wire wheel 207 to bend the active bend portion 106 of the catheter 104. Unless otherwise noted, the ureteroscope 400 may include any aspect of the ureteroscope 100. For example, the ureteroscope 400 may include any aspect of the control system 200 described above. In some embodiments, the steering controller 424 is configured to selectively bend the active bend portion 106 on one or more first axes and the control system 200 is configured to selectively bend the active bend portion 106 on one or more second axes different than the one or more first axes. In some embodiments, the ureteroscope 400 includes the steering controller 424, but the control system 200 is not configured control bending of the active bend portion 106 of the catheter 104. That is, the ureteroscope 400 may include a steering controller 424 to control bending of the active bend portion 106 of the catheter 104 and may include the control system 200 to control one or more of the light source 111, the image sensor of the optoelectronic module 109, the image collection mode of the image sensor, a frame grab, zoom of the image sensor, tool deployment in the working channel 152, and/or brightness, a zoom, a focus, or a contrast of the one or more images from the image sensor displayed on a display 190.

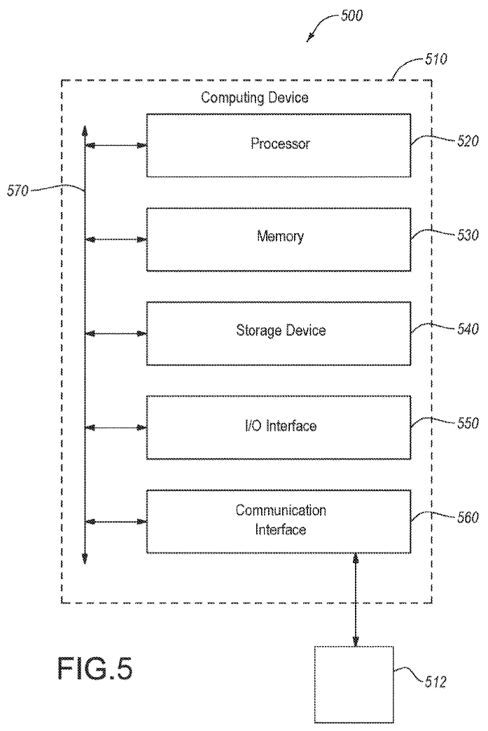

Systems and methods described herein may include use a controller to carry out one or more aspects of the systems and methods. For example, a controller may be used to carry out one or more acts performed by the processor in the systems and methods described herein. Moreover, one or more (e.g., all) of the processor 250, the communication interface 150 in the handpiece 110, the host machine 170, the computer 180, and/or the display 190 of the endoscopic system 10 may utilize or otherwise include a controller. For example, the processor 250 described in relation to the endoscopic system 10 may include or utilize a controller. FIG. 5 is a schematic of a controller 500 that may be utilized in any of the example systems and method described herein, according to an embodiment. The controller 500 may be configured to implement one or more acts of any of the example methods and systems disclosed herein. The controller 500 includes at least one computing device 510. The at least one computing device 510 is an exemplary computing device that may be configured to perform one or more of the acts described above, such as the act 415 of the method 400. The at least one computing device 510 can include one or more servers, one or more computers (e.g., desk-top computer, lap-top computer), or one or more mobile computing devices (e.g., smartphone, tablet, etc.). The computing device 510 can comprise at least one processor 520, memory 530, a storage device 540, an input/output (“I/O”) device/interface 550, and a communication interface 560. While an example computing device 510 is shown in FIG. 5, the components illustrated in FIG. 5 are not intended to be limiting of the controller 500 or computing device 510. Additional or alternative components may be used in some examples. Further, in some examples, the controller 500 or the computing device 510 can include fewer components than those shown in FIG. 5. For example, the controller 500 may not include the one or more additional computing devices 512. In some examples, the at least one computing device 510 may include a plurality of computing devices, such as a server farm, computational network, or cluster of computing devices. Components of computing device 510 shown in FIG. 5 are described in additional detail below.

In some examples, the processor(s) 520 includes hardware for executing instructions (e.g., instructions for carrying out one or more portions of any of the methods disclosed herein), such as those making up a computer program. For example, to execute instructions, the processor(s) 520 may retrieve (or fetch) the instructions from an internal register, an internal cache, the memory 530, or a storage device 540 and decode and execute them. As an example, the processor(s) 520 may include one or more instruction caches, one or more data caches, and one or more translation lookaside buffers (TLBs). Instructions in the instruction caches may be copies of instructions in memory 530 or storage device 540. In some examples, the processor 520 may be configured (e.g., include programming stored thereon or executed thereby) to carry out one or more portions of any of the example methods and systems disclosed herein.

In some examples, the processor 520 is configured to perform any of the acts disclosed herein and/or cause one or more portions of the computing device 510 or controller 500 to perform at least one of the acts disclosed herein. Such configuration can include one or more operational programs (e.g., computer program products) that are executable by the at least one processor 520. For example, the processor 520 may be configured to automatically determine receive data or signals from the sensor 202 and coordinate movement of the motor 206 to selectively bend the active bend portion 106.

The at least one computing device 510 (e.g., a server) may include at least one memory storage medium (e.g., memory 530 and/or storage device 540). The computing device 510 may include memory 530, which is operably coupled to the processor(s) 520. The memory 530 may be used for storing data, metadata, and programs for execution by the processor(s) 520. The memory 530 may include one or more of volatile and non-volatile memories, such as Random Access Memory (RAM), Read Only Memory (ROM), a solid state disk (SSD), Flash, Phase Change Memory (PCM), or other types of data storage. The memory 530 may be internal or distributed memory.

The computing device 510 may include the storage device 540 having storage for storing data or instructions. The storage device 540 may be operably coupled to the at least one processor 520. In some examples, the storage device 540 can comprise a non-transitory memory storage medium, such as any of those described above. The storage device 540 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 540 may include removable or non-removable (or fixed) media. Storage device 540 may be internal or external to the computing device 510. In some examples, storage device 540 may include non-volatile, solid-state memory. In some examples, storage device 540 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 530 and/or storage device 540 (e.g., memory storage medium(s)) may store one or more databases thereon.

In some examples, data from the one or more sensors of the endoscopic system 10, for example, may be stored in a memory storage medium such as one or more of the at least one processor 520 (e.g., internal cache of the processor), memory 530, or the storage device 540. In some examples, the at least one processor 520 may be configured to access (e.g., via bus 570) the memory storage medium(s) such as one or more of the memory 530 or the storage device 540. For example, the at least one processor 520 may receive and store the data (e.g., look-up tables) as a plurality of data points in the memory storage medium(s).

The computing device 510 also includes one or more I/O devices/interfaces 550, which are provided to allow a user to provide input to, receive output from, and otherwise transfer data to and from the computing device 510. These I/O devices/interfaces 550 may include a mouse, keypad or a keyboard, a touch screen, camera, optical scanner, network interface, web-based access, modem, a port, other known I/O devices or a combination of such I/O devices/interfaces 550. The touch screen may be activated with a stylus or a finger.

The I/O devices/interfaces 550 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 550 are configured to provide graphical data to a display for presentation to a user. The graphical data may be representative of one or more graphical user interfaces and/or any other graphical content as may serve a particular implementation.

The computing device 510 can further include a communication interface 560. The communication interface 560 can include hardware, software, or both. The communication interface 560 can provide one or more interfaces for communication (such as, for example, packet-based communication) between the computing device 510 and one or more additional computing devices 512 or one or more networks. For example, communication interface 560 may include a network interface controller (NIC) or network adapter for communicating with an Ethernet or other wire-based network or a wireless NIC (WNIC) or wireless adapter for communicating with a wireless network, such as a WI-FI.

Any suitable network and any suitable communication interface 560 may be used. For example, computing device 510 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 500 or computing device 510 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. Computing device 510 may include any suitable communication interface 560 for any of these networks, where appropriate.

The computing device 510 may include a bus 570. The bus 570 can include hardware, software, or both that couples components of computing device 510 to each other. For example, bus 570 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

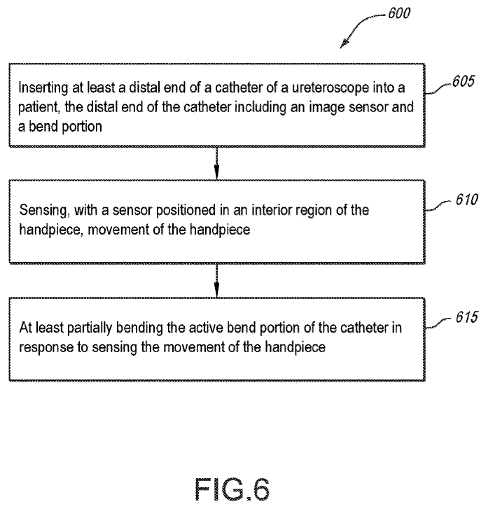

FIG. 6 is a flow diagram of a method 600 of operating a ureteroscope system, according to an embodiment. The method includes an act 605 of inserting at least a distal end of a catheter of a ureteroscope into a patient, the distal end of the catheter including an image sensor and a bend portion. The method includes an act 610 sensing, with a sensor positioned in an interior region of the handpiece, movement of the handpiece. The method includes an act 615 of at least partially bending the active bend portion of the catheter in response to sensing the movement of the handpiece. The ureteroscope may include any ureteroscope described herein. For example, the ureteroscope may include a motor positioned in the interior region of the handpiece and operably coupled to the sensor. The motor may be configured to be activated responsive to the sensor detecting movement of the handpiece, and one or more wires secured to the active bend portion of the catheter and adjustable responsive to activation of the motor effective to at least partially bend the active bend portion of the catheter. In some embodiments, the method may include an act of activating the catheter control system with a control positioned on the handpiece.

In some embodiments, the handpiece includes a working channel port positioned proximate to the distal end region and a cable port, and the catheter includes a working channel in fluid communication with the working channel port and an image sensor positioned at a distal end of the active bend portion distal to the handpiece. In these and other embodiments, the method 600 may further include an act of communicating with one or more electronic devices and providing power to at least one of the image sensor and the catheter control system with a cable that is connected to the handpiece at the cable port.

In some embodiments, the act 610 may include sensing, with the sensor, movement of the handpiece in a first handpiece direction and the act 615 may include at least partially bending the active bend portion in a first bending direction in response to sensing the movement of the handpiece in the first handpiece direction. In these and other embodiments, the method 600 may include an act of sensing, with the sensor, movement of the handpiece in a second handpiece direction and an act of at least partially bending the active bend portion in a second bending direction in response to sensing the movement of the handpiece in the second handpiece direction. The first bending direction may be substantially opposite to the first handpiece direction of movement of the handpiece and the second bending direction may be substantially opposite to the second handpiece direction of movement of the handpiece.

In some embodiments, the act 610 includes sensing a first magnitude of movement of the handpiece and the act 615 includes bending the active bend portion a first amount in response to sensing the first magnitude of movement of the handpiece. In these and other embodiments, the method 600 may include an act of sensing, with the sensor positioned in an interior region of the handpiece, a second magnitude of movement of the handpiece, the second magnitude of movement being greater than the first magnitude of movement. The method 600 also may include an act of bending the active bend portion a second amount in response to sensing the second magnitude of movement of the handpiece, the second amount of bending being greater than the first amount of bending.

In some embodiments, the method 600 further includes an act of at least partially bending the active bend portion of the catheter by moving a steering controller secured to the handpiece and positioned at least partially outside the interior region of the handpiece. In some embodiments, the method 600 further includes an act of sensing, with the sensor positioned in an interior region of the handpiece, movement of the handpiece in an additional direction. The method 600 may further include, in response to sensing the movement of the handpiece in the additional direction, activating or deactivating one or more of a light on the distal end of the catheter, the image sensor, an image collection mode of the image sensor, a frame grab to record a still image during a video stream mode, a zoom adjustment of the image sensor, a tool deployment in the working channel, and/or an adjustment at least one of a brightness, a zoom, a focus, or a contrast of the one or more images from the image sensor displayed on a display.

The act of the method 600 described above are for illustrative purposes. For example, the acts of the method 600 may be performed in different orders, split into multiple acts, modified, supplemented, or combined. In an embodiment, one or more of the acts of the method 600 may be omitted from the method 600. Any of the acts of the method 600 may include using any of the handpieces 110, ureteroscopes 100 or 400, or the system 10 disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

What is claimed is:

1. A ureteroscope system, comprising:
a handpiece having a proximal end region, a distal end region opposite to the proximal end region, and an interior region;
a catheter extending from the distal end region of the handpiece, the catheter including an active bend portion distal to the handpiece; and
a catheter control system including:
a sensor positioned in the interior region of the handpiece and configured to detect movement of the handpiece;
a motor positioned in the interior region of the handpiece and operably coupled to the sensor, the motor configured to be activated responsive to the sensor detecting movement of the handpiece; and
one or more wires secured to the active bend portion of the catheter and adjustable responsive to activation of the motor effective to at least partially bend the active bend portion of the catheter.

2. The ureteroscope system of claim 1, further comprising:
wherein the handpiece includes a working channel port positioned proximate to the distal end region and a cable port;
wherein the catheter includes a working channel in fluid communication with the working channel port and an image sensor positioned at a distal end of the active bend portion distal to the handpiece; and
a cable connected or connectable to the handpiece at the cable port and configured to provide power to at least one of the image sensor and the catheter control system and communicate with one or more electronic devices.

3. The ureteroscope system of any of claim 1 or 2, further comprising a control positioned on the handpiece and operably coupled to the catheter control system and configured to selectively activate and deactivate the catheter control system such that (1) when the control activates the catheter control system, the motor adjusts the one or more wires responsive to the sensor detecting movement of the handpiece and (2) when the control deactivates the catheter control system, the motor does not adjust the one or more wires responsive to the sensor detecting movement of the handpiece.

4. The ureteroscope system of any of claims 1-3, wherein:
the sensor is configured to detect movement of the handpiece in a first handpiece direction and a second handpiece direction different from the first handpiece direction;
the motor is configured to adjust the one or more wires responsive to movement of the handpiece in the first handpiece direction effective to bend the active bend portion in a first bending direction; and
the motor is configured to adjust the one or more wires responsive to movement of the handpiece in the second handpiece direction effective to bend the active bend portion in a second bending direction different from the first bending direction.

5. The ureteroscope system of claim 4, wherein:
the one or more wires include at least a first wire portion and a second wire portion each extending at least partially between the motor and the active bend portion;
the motor is configured adjust the first wire portion responsive to movement of the handpiece in the first handpiece direction effective to bend the active bend portion in the first bending direction; and
the motor is configured to adjust the second wire portion responsive to movement of the handpiece in the first handpiece direction effective to bend the active bend portion in the second bending direction.

6. The ureteroscope system of any of claim 4 or 5, wherein the first bending direction is substantially opposite to the first handpiece direction of movement of the handpiece and the second bending direction is substantially opposite to the second handpiece direction of movement of the handpiece.

7. The ureteroscope system of any of claims 1-6, wherein:
the sensor is configured to detect a magnitude of movement of the handpiece; and
the motor is configured to adjust the one or more wires approximately proportionally to the magnitude of movement of the handpiece such that the active bend portion bends proportionally to the magnitude of movement of the handpiece.

8. The ureteroscope system of any of claims 1-7, wherein the catheter control system includes a driver operably coupled tot the sensor and the motor.

9. The ureteroscope system of claim 8, wherein the driver includes a stepper driver and the motor includes a stepper motor.

10. The ureteroscope system of any of claims 1-9, further comprising a wire wheel secured to the interior region of the handpiece and the motor, wherein one or more wires are secured to the wire wheel and the motor is configured to rotate the wire wheel in one or more directions effective to adjust the one or more wires responsive to movement detected by the sensor.

11. The ureteroscope system of claim 10, further comprising a steering controller secured to the handpiece and positioned at least partially outside the interior region of the handpiece, the steering controller operably secured to the wire wheel and selectively movable effective to move the wire wheel and adjust the one or more wires secured to the wire wheel and bend the active bend portion of the catheter.

12. The ureteroscope system of any of claims 2-11, further comprising a light at the distal end of the active bend portion, wherein the sensor is configured to detect movement of the handpiece in an additional direction effective to activate or deactivate the light, activate or deactivate the image sensor, change an image collection mode of the image sensor from a still image mode to a video stream mode, activate a frame grab to record a still image during the video stream mode, adjust zoom of the image sensor, deploy a tool in the working channel, and/or adjust at least one of a brightness, a zoom, a focus, or a contrast of the one or more images from the image sensor displayed on a display.

13. A method of operating a ureteroscope system, the method comprising:
inserting at least a distal end of a catheter of a ureteroscope into a patient, the distal end of the catheter including an image sensor and an active bend portion;
sensing, with a sensor positioned in an interior region of the handpiece, movement of the handpiece; and
at least partially bending the active bend portion of the catheter in response to sensing the movement of the handpiece, the ureteroscope including a motor positioned in the interior region of the handpiece and operably coupled to the sensor, the motor configured to be activated responsive to the sensor detecting movement of the handpiece, and one or more wires secured to the active bend portion of the catheter and adjustable responsive to activation of the motor effective to at least partially bend the active bend portion of the catheter.

14. The method of claim 13, further comprising:
wherein the handpiece includes a working channel port positioned proximate to the distal end region and a cable port;
wherein the catheter includes a working channel in fluid communication with the working channel port and an image sensor positioned at a distal end of the active bend portion distal to the handpiece; and
communicating with one or more electronic devices and providing power to at least one of the image sensor and the catheter control system with a cable that is connected to the handpiece at the cable port.

15. The method of any of claim 13 or 14, further comprising activating the catheter control system with a control positioned on the handpiece.

16. The method of any of claims 13-15, further comprising:
wherein sensing, with a sensor positioned in an interior region of the handpiece, movement of the handpiece includes sensing, with the sensor, movement of the handpiece in a first handpiece direction;
wherein at least partially bending the active bend portion of the catheter in response to sensing the movement of the handpiece includes at least partially bending the active bend portion in a first bending direction in response to sensing the movement of the handpiece in the first handpiece direction;
sensing, with the sensor, movement of the handpiece in a second handpiece direction; and
at least partially bending the active bend portion in a second bending direction in response to sensing the movement of the handpiece in the second handpiece direction.

17. The method of claim 16, wherein the first bending direction is substantially opposite to the first handpiece direction of movement of the handpiece and the second bending direction is substantially opposite to the second handpiece direction of movement of the handpiece.

18. The method of any of claims 13-17, further comprising:
wherein sensing, with a sensor positioned in an interior region of the handpiece, movement of the handpiece includes sensing a first magnitude of movement of the handpiece;
wherein at least partially bending the active bend portion of the catheter in response to sensing the movement of the handpiece includes bending the active bend portion a first amount in response to sensing the first magnitude of movement of the handpiece;
sensing, with the sensor positioned in an interior region of the handpiece, a second magnitude of movement of the handpiece, the second magnitude of movement being greater than the first magnitude of movement; and
bending the active bend portion a second amount in response to sensing the second magnitude of movement of the handpiece, the second amount of bending being greater than the first amount of bending.

19. The method of any of claims 13-18, further comprising at least partially bending the active bend portion of the catheter by moving a steering controller secured to the handpiece and positioned at least partially outside the interior region of the handpiece.

20. The method of any of claims 13-19, further comprising:
sensing, with the sensor positioned in an interior region of the handpiece, movement of the handpiece in an additional direction; and
in response to sensing the movement of the handpiece in the additional direction, activating or deactivating one or more of a light on the distal end of the catheter, the image sensor, an image collection mode of the image sensor, a frame grab to record a still image during a video stream mode, a zoom adjustment of the image sensor, a tool deployment in the working channel, and/or an adjustment at least one of a brightness, a zoom, a focus, or a contrast of the one or more images from the image sensor displayed on a display.
